(19) United States
(12) Patent Application Publication
(10) Pub. No.: US 2011/0071199 A1
(43) Pub. Date: Mar. 24, 2011

(54) Thiophenyl Sulfonamides for the Treatment of Alzheimer's Disease

(75) Inventor: John E. Starrett, JR., Middletown, CT (US)

(73) Assignee: Bristol-Myers Squibb Company

(21) Appl. No.: 12/724,949

(22) Filed: Mar. 16, 2010

Related U.S. Application Data
(60) Provisional application No. 61/161,801, filed on Mar. 20, 2009.

(51) Int. Cl. A61K 31/4245; C07D 333/34; C07D 413/12; A61K 31/381; A61K 31/422; A61P 25/28; A61P 25/00

(52) U.S. Cl. 514/364; 549/65; 548/131; 548/235; 514/445; 514/374

(57) ABSTRACT

There is provided a series of novel α-(N-sulfonamido)acetamide compounds of the Formula (I)

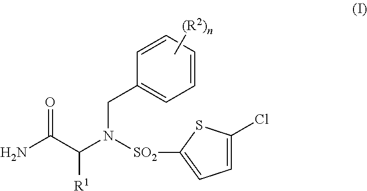

wherein R¹, R² and n are defined herein, which are inhibitors of β-amyloid peptide (β-AP) production and are useful in the treatment of Alzheimer's Disease and other conditions affected by anti-amyloid activity.

### CROSS-REFERENCE TO RELATED APPLICATIONS

This application claims the benefit of U.S. Provisional Application Ser. No. 61/161,801 filed Mar. 20, 2009.

### FIELD OF THE INVENTION

This invention provides novel thiophenyl sulfonamide compounds having drug and bio-affecting properties, their pharmaceutical compositions and method of use. In particular, the invention is concerned with thiophenyl sulfonamides. These compounds possess unique inhibition of the β-amyloid peptide (β-AP) production, thereby acting to prevent the accumulation of amyloid protein deposits in the brain. More particularly, the present invention relates to the treatment of Alzheimer's Disease (AD). Additionally, the present invention is useful in the treatment of head trauma, traumatic brain injury, dementia pugilistica, and/or other conditions associated with β-amyloid peptide.

### BACKGROUND OF THE INVENTION

Alzheimer's disease (AD) is a progressive neurodegenerative disease which begins with memory loss and progresses to include severe cognitive impairment, altered behavior, and decreased motor function (Grundman, M. et al., Arch Neurol. (2004) 61: 59-66; Walsh, D. M. et al., Neuron (2004) 44: 181-193). It is the most common form of dementia and represents the third leading cause of death after cardiovascular disorders and cancer. The cost of AD is enormous and includes the suffering of the patients and families and the lost productivity of patients and caregivers. No treatment that effectively prevents AD or reverses the clinical symptoms and underlying pathophysiology is currently available.

A definitive diagnosis of AD for a demented patient requires a histopathological evaluation of the number and localization of neuritic plaques and neurofibrillary tangles upon autopsy (Consensus recommendations for the postmortem diagnosis of Alzheimer's disease. Neurobiol Aging (1997) 18: S1-2). Similar alterations are observed in patients with Trisomy 21 (Down syndrome). Plaques primarily consist of β-amyloid (Aβ) peptides that are formed by a stepwise proteolytic cleavage of the amyloid precursor protein (APP) by β-site APP-cleaving enzyme (BACE), to generate the N-terminus, and γ-secretase, to generate the C-terminus (Selkoe, D J., Physiol Rev. (2001) 81: 741-766). γ-Secretase is a transmembrane protein complex that includes Nicastrin, Aph-1, PEN-2, and either Presenilin-1 (PS-1) or Presenilin-2 (PS-2) (Wolfe, M. S. et al., Science (2004) 305: 1119-1123). PS-1 and PS-2 are believed to contain the catalytic sites of γ-secretase.

Aβ40 is the most abundant form of Aβ synthesized (80-90%), while Aβ42 is most closely linked with AD pathogenesis. In particular, mutations in the APP, PS-1, and PS-2 genes that lead to rare, familial forms of AD implicate Aβ42 aggregates as the primary toxic species (Selkoe, D. J., Physiol Rev., (2001) 81: 741-766). Current evidence suggests that oligomeric, protofibrillar and intracellular Aβ42 play a significant role in the disease process (Cleary, J. P. et al., Nat Neurosci. (2005) 8: 79-84). Inhibitors of the enzymes that form Aβ42, such as γ-secretase, represent potential disease-modifying therapeutics for the treatment of AD.

γ-Secretase cleaves multiple type I transmembrane proteins in addition to APP (Pollack, S. J. et al., Curr Opin Investig Drugs (2005) 6: 35-47). While the physiological significance of most of these cleavage events is unknown, genetic evidence indicates that γ-secretase cleavage of Notch is required for Notch signaling (Artavanis-Tsakonas, S. et al., Science (1999) 284(5415): 770-6; Kadesch, T.; Exp Cell Res. (2000) 260(1): 1-8). In rodents dosed with γ-secretase inhibitors, drug-related toxicity has been identified in the gastrointestinal (GI) tract, thymus, and spleen (Searfoss, G. H.; Jordan et al., J Biol Chem. (2003) 278: 46107-46116; Wong, G. T. et al., J Biol Chem. (2004) 279: 12876-12882; Milano, J. et al., Toxicol Sci. (2004) 82: 341-358). These toxicities are likely linked to inhibition of Notch signaling (Jensen, J. et al., Nat Genet. (2000) 24: 36-44).

The identification of mechanism-based toxicity raises the question of whether an acceptable therapeutic index can be achieved with γ-secretase inhibitors. Selective inhibition of Aβ formation over Notch processing, pharmacokinetics, drug disposition and/or tissue-specific pharmacodynamics could impact therapeutic margin.

Evidence suggests that a reduction in brain Aβ levels by inhibition of γ-secretase may prevent the onset and progression of AD (Selkoe, D. Physiol. Rev. (2001) 81: 741-766; Wolfe, M., J. Med. Chem. (2001) 44: 2039-2060). There are emerging data for the role of Aβ in other diseases, including mild cognitive impairment (MCI), Down syndrome, cerebral amyloid angiopathy (CAA), dementia with Lewy bodies (DLB), amyotrophic lateral sclerosis (ALS-D), inclusion body myositis (IBM), and age-related macular degeneration. Advantageously, compounds that inhibit γ-secretase and reduce production of Aβ could be used to treat these or other Aβ-dependent diseases.

Excess production and/or reduced clearance of Aβ causes CAA (Thal, D. et al., J. Neuropath. Exp. Neuro. (2002) 61: 282-293). In these patients, vascular amyloid deposits cause degeneration of vessel walls and aneurysms that may be responsible for 10-15% of hemorrhagic strokes in elderly patients. As in AD, mutations in the gene encoding Aβ lead to an early onset form of CAA, referred to as cerebral hemorrhage with amyloidosis of the Dutch type, and mice expressing this mutant protein develop CAA that is similar to patients. Compounds that specifically target γ-secretase could reduce or prevent CAA.

DLB manifests with visual hallucinations, delusions, and parkinsonism. Interestingly, familial AD mutations that cause Aβ deposits can also cause Lewy bodies and DLB symptoms (Yokota, O. et al., Acta Neuropathol (Berl) (2002) 104: 637-648). Further, sporadic DLB patients have Aβ deposits similar to those in AD (Deramecourt, V. et al., J Neuropathol Exp Neural (2006) 65: 278-288). Based on this data, Aβ likely drives Lewy body pathology in DLB and, therefore, γ-secretase inhibitors could reduce or prevent DLB.

Approximately 25% of ALS patients have significant dementia or aphasia (Hamilton, R. L. et al., Acta Neuropathol (Berl) (2004) 107: 515-522). The majority (˜60%) of these patients, designated ALS-D, contain ubiquitin-positive inclusions comprised primarily of the TDP-43 protein (Neumann, M. et al., Science (2006) 314:

130-133). About 30% of the ALS-D patients have amyloid plaques consistent with Aβ causing their dementia (Hamilton, R. L. et al., Acta Neuropathol (Berl) (2004) 107: 515-522). These patients should be identifiable with amyloid imaging agents and potentially treatable with γ-secretase inhibitors.

IBM is a rare, age-related degenerative disease of skeletal muscle. The appearance of Aβ deposits in IBM muscle and the recapitulation of several aspects of the disease by directing APP overexpression to muscle in transgenic mice support the role of Aβ in IBM (reviewed in Murphy, M. P. et al., Neurology (2006) 66: 565-68). Compounds that specifically target γ-secretase could reduce or prevent IBM.

In age-related macular degeneration, Aβ was identified as one of several components of drusen, extracellular deposits beneath the retinal pigment epithelium (RPE) (Anderson, D. H. et al., Exp Eye Res (2004) 78: 243-256). A recent study has shown potential links between Aβ and macular degeneration in mice (Yoshida, T. et al., J Clin Invest (2005) 115: 2793-2800). Increases in Aβ deposition and supranuclear cataracts have been found in AD patients (Goldstein, L. E. et al., Lancet (2003) 361: 1258-1265). Compounds that specifically target γ-secretase could reduce or prevent age-related macular degeneration.

Based on the role of Notch signaling in tumorigenesis, compounds which inhibit γ-secretase may also be useful as therapeutic agents for the treatment of cancer (Shih, I.-M., et al., Cancer Research (2007) 67: 1879-1882).

Compounds which inhibit gamma secretase may also be useful in treating conditions associated with loss of myelination, for example multiple sclerosis (Watkins, T A., et al., Neuron (2008) 60: 555-569).

A recent study by Georgetown University Medical Center researchers suggests that gamma-secretase inhibitors may prevent long-term damage from traumatic brain injury (Loane, D. J., et al., Nature Medicine (2009): 1-3).

### SUMMARY OF THE INVENTION

A series of thiophenyl sulfonamide derivatives have been synthesized. These compounds specifically inhibit the production of β-amyloid peptide (β-AP) from β-amyloid precursor protein (β-APP). The pharmacologic action of these compounds makes them useful for treating conditions responsive to the inhibition of β-AP in a patient; e.g., Alzheimer's Disease (AD), and Down's Syndrome, head trauma, traumatic brain injury, and dementia pugilistica,. Therapy utilizing administration of these compounds to patients suffering from, or susceptible to, these conditions involves reducing β-AP available for accumulation and deposition in brains of these patients.

### DETAILED DESCRIPTION OF THE INVENTION

The present invention comprises compounds of Formula I, their pharmaceutical formulations, and their use in inhibiting β-AP production in patients suffering from or susceptible to AD or other disorders resulting from β-AP accumulation in brain tissue. The compounds of Formula I which include salts, nontoxic pharmaceutically acceptable salts and/or hydrates thereof have the following formula and meanings:

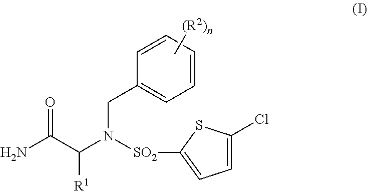

wherein:
R¹ is C₁₋₆ alkyl, —(CH₂)ᵣ—C₃₋₆ cycloalkyl, or C₁₋₆ haloalkyl;
R² is halo, oxadiazolyl, or oxazolyl;
n is 1 to 3; and
r is 1 to 3.

In another embodiment, the present invention provides for compounds of formula (I) or salts thereof, wherein the compound is of formula (Ia) or salt thereof

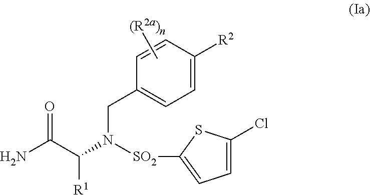

R² is halo, oxadiazolyl, or oxazolyl;
R^{2a} is halo;
n is 0-12.

In another embodiment, the present invention provides for compounds of formula (I) or salts thereof, wherein R¹ is C₁₋₄ alkyl, —(CH₂)ᵣ—C₃₋₆ cycloalkyl, or C₁₋₃ haloalkyl.

In another embodiment, the present invention provides for compounds of formula (I) or salts thereof, wherein R¹ is —(CH₂)—C₃₋₆ cycloalkyl, or C₁₋₃ haloalkyl.

In another embodiment, the present invention provides for compounds of formula (I) or salts thereof; wherein R² is halo,

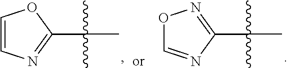

In another embodiment, the present invention provides for compounds of formula (I) or salts thereof, wherein R¹ is —(CH₂)-cyclopropyl, or —CH₂—CH₂—CF₃.

In another embodiment, the present invention provides for compounds of formula (I) or salts thereof,

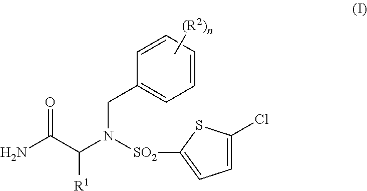

wherein:
R¹ is cyclopropylmethyl, or CF₃—CH₂—CH₂—;
(cycloalkyl —CH2-) or haloalkyl;
R² is halo, oxadiazolyl, or oxazolyl;
n is 1 to 3.

In another embodiment, the present invention provides for compounds of formula (I) or salts thereof, wherein the compound is of formula (Ia)

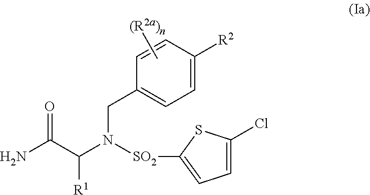

R² is halo, oxadiazolyl, or oxazolyl;
R^{2a} is halo;
n is 0-1.

In another embodiment, the present invention provides for compounds of formula (I) or salts thereof, R² is halo,

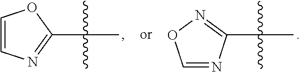

In another embodiment, the present invention provides for compounds of formula (I) or salts thereof, wherein the compound is of formula (Ib)

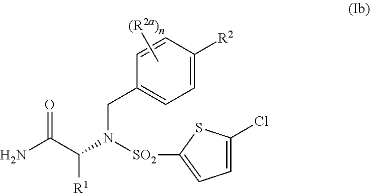

The present invention also provides for a pharmaceutical composition comprising a compound of formula (I) in association with a pharmaceutically acceptable adjuvant, carrier or diluent.

The present invention also provides a method for the treatment or alleviation of disorders associated with β-amyloid peptide, especially Alzheimer's Disease, which comprises administering together with a conventional adjuvant, carrier or diluent a therapeutically effective amount of a compound of formula I or a nontoxic pharmaceutically acceptable salt, solvate or hydrate thereof.

In another embodiment, the present invention provides a compound of the present invention for use in therapy.

In another embodiment, the present invention provides a compound of the present invention for use in therapy for treating or delaying the onset of Alzheimer's disease, cerebral amyloid angiopathy, mild cognitive impairment and/or Down syndrome. In another embodiment, the present invention also provides the use of a compound of formula I of the present invention for the manufacture of a medicament for the treatment or delaying the onset of Alzheimer's disease, cerebral amyloid angiopathy, mild cognitive impairment and/or Down syndrome.

In yet another embodiment, this invention relates to a method for the treatment of head trauma, traumatic brain injury, and/or dementia pugilistica, which comprises administering to a mammal in need thereof a therapeutically effective amount of the compound of Formula I or a solvate or hydrate thereof.

In yet another embodiment, this invention relates to a method for the treatment of head trauma which comprises administering to a mammal in need thereof a therapeutically effective amount of the compound of Formula I or a solvate or hydrate thereof.

In yet another embodiment, this invention relates to a method for the treatment of traumatic brain injury which comprises administering to a mammal in need thereof a therapeutically effective amount of the compound of Formula I or a solvate or hydrate thereof.

In yet another embodiment, this invention relates to a method for the treatment of dementia pugilistica which comprises administering to a mammal in need thereof a therapeutically effective amount of the compound of Formula I or a solvate or hydrate thereof.

The present invention may be embodied in other specific forms without departing from the spirit or essential attributes thereof. This invention encompasses all combinations of preferred aspects and/or embodiments of the invention noted herein. It is understood that any and all embodiments of the present invention may be taken in conjunction with any other embodiment or embodiments to describe additional more preferred embodiments. It is also to be understood that each individual element of the preferred embodiments is its own independent preferred embodiment. Furthermore, any element of an embodiment is meant to be combined with any and all other elements from any embodiment to describe an additional embodiment.

### DEFINITIONS

The following are definitions of terms that may be used in the present specification. The initial definition provided for a group or term herein applies to that group or term throughout the present specification individually or as part of another group, unless otherwise indicated.

The term “C₁₋₆ alkyl” as used herein and in the claims (unless the context indicates otherwise) means straight or branched chain alkyl groups such as methyl, ethyl, propyl, isopropyl, butyl, isobutyl, t-butyl, pentyl, 3-methylbutyl, hexyl and the like. The term “C₂₋₆ alkenyl” used herein and in the claims (unless the context indicates otherwise) means straight or branched chain alkenyl groups such as ethenyl (i.e. vinyl), propenyl, allyl, butenyl, 3-methylbutenyl, pentenyl, hexenyl and the like. Unless otherwise specified, the term “halogen” as used herein and in the claims is intended to include bromine, chlorine, iodine and fluorine while the term “halide” is intended to include bromide, chloride and iodide anion.

The term “C₃₋₇ cycloalkyl” means a carbon cyclic ring system such as cyclopropyl, cyclobutyl, cyclopentyl, cyclohexyl and cycloheptyl.

The term “C₁₋₄ haloalkyl” means a straight or branched chain C₁₋₄ alkyl group containing from 1 to 3 halogen atoms such as trifluoromethyl, fluoroethyl, 1,2-dichloroethyl, trichloroethyl and the like.

When any variable (e.g., R²) occurs more than one time in any constituent or formula for a compound, its definition at each occurrence is independent of its definition at every other occurrence. Thus, for example, if a group is shown to be substituted with 0-2 R², then said group may optionally be substituted with up to two R² groups and R² at each occurrence is selected independently from the definition of R². Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

The compounds of formula I may exist as a free form {with no ionization) or may form salts which are also within the scope of this invention. Pharmaceutically acceptable (i.e. non-toxic, physiologically acceptable) salts are preferred, although other salts are also useful, e.g., in isolating or purifying the compounds of this invention.

The compounds of formula I may form salts with alkali metals such as sodium, potassium and lithium, with alkaline earth metals such as calcium and magnesium, with organic bases such as dicyclohexylamine, tributylamine, pyridine and amino acids such as arginine, lysine and the like. Such salts can be formed as known to those skilled in the art.

The compounds for formula I may form salts with a variety of organic and inorganic acids. Such salts include those formed with hydrogen chloride, hydrogen bromide, methanesulfonic acid, sulfuric acid, acetic acid, trifluoroacetic acid, oxalic acid, maleic acid, benzenesulfonic acid, toluenesulfonic acid and various others (e.g., nitrates, phosphates, borates, tartrates, citrates, succinates, benzoates, ascorbates, salicylates and the like). Such salts can be formed as known to those skilled in the art.

In addition, zwitterions (“inner salts”) may be formed.

All stereoisomers of the compounds of the instant invention are contemplated, either in admixture or in pure or substantially pure form. The definition of compounds according to the invention embraces all the possible stereoisomers and their mixtures. It very particularly embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates from the conventional methods, such as, for example, salt formation with an optically active acid followed by crystallization.

The present invention is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include 13C and 14C. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

It should further be understood that solvates (e.g., hydrates) of the compounds of formula I are also with the scope of the present invention. Methods of solvation are generally known in the art.

“Stable compound” and “stable structure” are meant to indicate a compound that is sufficiently robust to survive isolation to a useful degree of purity from a reaction mixture, and formulation into an efficacious therapeutic agent. It is preferred that there presently recited compounds do not contain a N-halo, S(O)₂H, or S(O)H group.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting it development; and/or (c) relieving the disease-state, i.e., causing regression of the disease state.

“Therapeutically effective amount” is intended to include an amount of a compound of the present invention that is effective when administered alone or in combination. “Therapeutically effective amount” is also intended to include an amount of the combination of compounds which is effective for the treatment of disease.

The present invention further includes compositions comprising one or more compounds of the present invention and a pharmaceutically acceptable carrier.

A “pharmaceutically acceptable carrier” refers to media generally accepted in the art for the delivery of biologically active agents to animals, in particular, mammals. Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage foam. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Remington's Pharmaceutical Sciences, 17th ed., Mack Publishing Company, Easton, Pa., 1985, which is incorporated herein by reference in its entirety.

As the compounds of the present invention possess an asymmetric carbon atom, the present invention includes the racemate as well as the individual enantiometric forms of the compounds of Formula I as described herein and in the claims. The use of a single designation such as (R) or (S) is intended to include mostly one stereoisomer. Mixtures of isomers can be separated into individual isomers according to methods which are known per se, e.g. fractional crystallization, adsorption chromatography or other suitable separation processes. Resulting racemates can be separated into antipodes in the usual manner after introduction of suitable salt-forming groupings, e.g. by forming a mixture of diastereosiomeric salts with optically active salt-forming agents, separating the mixture into diastereomeric salts and converting the separated salts into the free compounds. The possible enantiomeric forms may also be separated by fractionation through chiral high pressure liquid chromatography columns.

The term “nontoxic pharmaceutically acceptable salt” as used herein and in the claims is intended to include nontoxic base addition salts. Suitable salts include those derived from organic and inorganic acids such as, without limitation, hydrochloric acid, hydrobromic acid, phosphoric acid, sulfuric acid, methanesulfonic acid, acetic acid, tartaric acid, lactic acid, sulfinic acid, citric acid, maleic acid, fumaric acid, sorbic acid, aconitic acid, salicylic acid, phthalic acid, and the like.

In the method of the present invention, the term “therapeutically effective amount” means the total amount of each active component of the method that is sufficient to show a meaningful patient benefit, i.e., healing of acute conditions characterized by inhibition of β-amyloid peptide production. When applied to an individual active ingredient, administered alone, the term refers to that ingredient alone. When applied to a combination, the term refers to combined amounts of the active ingredients that result in the therapeutic effect, whether administered in combination, serially or simultaneously. The terms “treat, treating, treatment” as used herein and in the claims means preventing or ameliorating diseases associated with β-amyloid peptide.

### Utility

For therapeutic use, the pharmacologically active compound of Formula I will normally be administered as a pharmaceutical composition comprising as the (or an) essential active ingredient at least one such compound in association with a solid or liquid pharmaceutically acceptable carrier and, optionally, with pharmaceutically acceptable adjuvants and excipients employing standard and conventional techniques.

The pharmaceutical compositions include suitable dosage forms for oral, parenteral (including subcutaneous, intramuscular, intradermal and intravenous), transdermal, sublingual, bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques. Oral preparations include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. For topical or nasal administration, penetrants or permeation agents that are appropriate to the particular barrier to be permeated are used in the formulation. Such penetrants are generally known in the art. The pharmaceutical compositions are prepared by conventional techniques appropriate to the desired preparation containing appropriate amounts of the active ingredient, that is, the compound of Formula I according to the invention. See, for example, Remington's Pharmaceutical Sciences, Mack Publishing Company, Easton, Pa., 17th edition, 1985.

The dosage of the compound of Formula I to achieve a therapeutic effect will depend not only on such factors as the age, weight and sex of the patient and mode of administration, but also on the degree of Aβ inhibition desired and the potency of the compound of Formula I for the particular disorder or disease concerned. It is also contemplated that the treatment and dosage of the compound of Formula I may be administered in unit dosage form and that the unit dosage form would be adjusted accordingly by one skilled in the art to reflect the relative level of activity. The decision as to the particular dosage to be employed (and the number of times to be administered per day) is within the discretion of the physician, and may be varied by titration of the dosage to the particular circumstances of this invention to produce the desired therapeutic effect.

A suitable dose of the compound of Formula I or pharmaceutical composition thereof for a mammal, including man, suffering from, or likely to suffer from any condition related to Aβ peptide production as described herein, generally the daily dose will be from about 0.01 mg/kg to about 10 mg/kg and preferably, about 0.1 to 2 mg/kg when administered parenterally. For oral administration, the dose may be in the range from about 0.01 to about 20 mg/kg and preferably from 0,1 to 10 mg/kg body weight. The active ingredient will preferably be administered in equal doses from one to four times a day. However, usually a small dosage is administered, and the dosage is gradually increased until the optimal dosage for the host under treatment is determined. In accordance with good clinical practice, it is preferred to administer the instant compound at a concentration level that will produce an effective anti-amyloid effect without causing any harmful or untoward side effects. However, it will be understood that the amount of the compound actually administered will be determined by a physician, in the light of the relevant circumstances including the condition to be treated, the choice of compound of be administered, the chosen route of administration, the age, weight, and response of the individual patient, and the severity of the patient's symptoms.

### Biological Testing Methods

Compounds of Formula (I) are expected to possess γ-secretase inhibitory activity. The detection of γ-secretase activity requires assays capable of reliable, accurate and expedient detection of γ-secretase cleavage products, particularly Aβ. The γ-secretase inhibitory activity of the compounds of the present invention is demonstrated using assays for such activity, for example, using the assays described below. Compounds within the scope of the present invention have been shown to inhibit the activity of γ-secretase, as determined using assays for such activity.

Compounds provided by this invention should also be useful as standards and reagents in determining the ability of a potential pharmaceutical to inhibit Aβ production. These would be provided in commercial kits comprising a compound of this invention.

### Inhibition of Aβ Formation in Cultured Cells

Compounds were assayed for Aβ40 or Aβ42 inhibition in cells using H4 APP751 SWE clone 8.20, developed at Bristol-Myers Squibb, an H4 neuroglioma cell line stably expressing the Swedish mutant of APP751. Cells were maintained in log phase through twice weekly passage at a 1:20 dilution. For IC₅₀ determinations, 30 μL cells (1.5×10⁴ cells/well) in DMEM media containing 0.0125% BSA (Sigma A8412) were plated directly into 384-well compound plates (Costar 3709) containing 0.1 μL serially diluted compound in DMSO. Following incubation for 19 hours in 5% CO₂ at 37° C., plates were briefly centrifuged (1000 rpm, 5 min). A 10 μL aliquot from each well was transferred to a second assay plate (Costar 3709) for Aβ40 measurements. Antibody cocktails were freshly prepared by dilution into 40 mM Tris-HCl (pH 7.4) with 0.2% BSA and added to assay plates. For Aβ42 measurements, antibodies specific for the Aβ42 neoepitope (565, developed at Bristol-Myers Squibb; conjugated to the Wallac reagent (Perkin Elmer)) and the N-terminal sequence of Aβ peptide (26D6, developed at SIBIA/Bristol-Myers Squibb; conjugated to APC (Perkin Elmer)) were mixed and 20 ∞L of the mixture was added to each well of the incubated cell plate yielding a final concentration of 0.8 ng/well 565 and 75 ng/well 26D6. For the Aβ40 measurements, antibodies specific for the Aβ40 neoepitope (TSD, developed at Bristol-Myers Squibb; conjugated to the Wallac reagent (Perkin Elmer)) and 26D6 as described above were mixed and 20 μL of the mixture was added to the 10 μL aliquots which had been removed previously from the cell plate yielding a final concentration of 1.6 ng/well TSD and 17.5 ng/well 26D6. Assay plates containing antibodies were sealed with aluminum foil and incubated overnight at 4° C. Signal was determined using a Viewlux counter (Perkin Elmer) and IC₅₀ values determined using curve fitting in CurveMaster (Excel Fit based).

TABLE 1
Examples of activity in the in vitro assay based on the inhibition of Aβ
formation in cultured cells

| EXAMPLE | IC₅₀ |
|---|---|
| 1 | 1.01 |
| 2 | 0.52 |
| 3 | 0.76 |
| 4 | 0.82 |
| 5 | 0.51 |
| 6 | 1.49 |
| 7 | 0.29 |
| 9 | 0.61 |
| 10 | 2.54 |
| 11 | 1.06 |
| 12 | 2.98 |

Compounds of the present invention have been demonstrated to have an IC₅₀ value less than 10 μM in one or all of the above assays. Therefore, the compounds of Formula I or pharmaceutical compositions thereof are useful in the treatment, alleviation or elimination of disorders or other disorders associated with the inhibition of β-amyloid peptide.

In addition to cleaving APP, γ-secretase cleaves other substrates, including: the Notch family of transmembrane receptors (reviewed in: Selkoe, D. Physiol. Rev. 2001, 81, 741-766; Wolfe, M. J. Med. Chem. 2001 44, 2039-2060); LDL receptor-related protein (May, P., Reddy, Y. K., Herz, J. J. Biol. Chem. 2002, 277, 18736-18743); ErbB-4 (Ni, C. Y., Murphy, M. P., Golde, T. E., Carpenter, G. Science 2001, 294, 2179-2181); E-cadherin (Marambaud, P., Shioi, J., et al., EMBO J. 2002, 21,1948-1956); and CD44 (Okamoto, I., Kawano, Y., et al., J. Cell Biol. 2001, 155, 755-762). If inhibition of cleavage of non-APP substrates causes undesirable effects in humans, then desired γ-secretase inhibitors would preferentially inhibit APP cleavage relative to unwanted substrates. Notch cleavage can be monitored directly by measuring the amount of cleavage product or indirectly by measuring the effect of the cleavage product on transcription (Mizutani, T., Taniguchi, Y., et al. Proc. Natl. Acad. Sci. USA 2001, 98, 9026-9031).

### In Vivo Pharmacology

### Abeta ELISAs

Aβ species from animals were measured using sandwich ELISA assays. A brief discussion of these assays is included here since the details of the epitopes for the individual antibodies determines the Aβ species that are detected. Mouse and rat Aβ share a common Aβ sequence that differs from human Aβ. As a result of these sequence differences, antibodies that recognize the N-terminal region of human Aβ, such as 26D6, bind weakly to rodent Aβ. Likewise, antibodies that bind tightly to rodent Aβ, such as 252, bind weakly to human Aβ. Two assays were developed for measuring rodent Aβ40: 252-TSD and 4G8-TSD. The TSD-4G8 assay can measure not only Aβ40, but other BACE-γ-secretase cleavage products (Aβ11-40) and α-secretase-γ-secretase cleavage products (P3). Table 2 summarizes the assays presented in this application and their use.

TABLE 2
Summary of Antibody Pairs Used to Assay In Vivo Samples

| Antibody Pair | Tissues analyzed | Aβ species detected |
|---|---|---|
| 252-TSD | Rat brain | Aβx-40^{a} |
| 4G8-TSD | Rat plasma, CSF | Aβx-40 & P3 |

^{a}The exact location of “x” is unknown. While 252 recognizes the N-terminal region of Aβ, it is unknown whether amino terminal truncation of Aβ affects 252 binding. This uncertainty is unlikely to be an issue in rats since N-terminal truncation is rare.

Each of these assays was validated using several methods. First, varying amounts of synthetic Aβ were added to the biological matrix and the increase in signal was compared to that obtained with synthetic Aβ in buffer solution. Second, Aβ was depleted from the biological sample with anti-Aβ antibodies. Third, samples were assayed from animals that were treated with high doses of a γ-secretase inhibitor. A validated assay efficiently detected exogenously added Aβ (>80% recovery), had a greatly reduced signal after Aβ immunodepletion (>80% reduction compared to nonspecific controls), and had a signal reduced to values approaching or overlapping with the assay floor using samples from animals treated with high doses of a γ-secretase inhibitor. The optimized and validated Aβ assays still contained a small amount of the signal (5-20% of vehicle control) which could not be depleted by anti-Aβ antibodies or treatment with γ-secretase inhibitors. This signal is unlikely to be Aβ and is consequently referred to as the assay floor. The assay floor was not used to correct any of the Aβ measurements and consequently, the values reported are likely underestimates of the actual amount of Aβ inhibition.

Aβ40 was used as a surrogate for Aβ42 in vivo. Aβ40 is approximately 10-fold more abundant in biological samples than Aβ42. Aβ40 is a good surrogate for Aβ42 based on experiments in cultured cells where Aβ40 and Aβ42 were similarly inhibited by the Compound of Example 7 and other γ-secretase inhibitors.

### Rat Studies

### In Life

Female Harlan Sprague-Dawley rats (˜200-250 g) were dosed daily by oral gavage with a dosing vehicle of 99% PEG-400, 1% Tween-80 at 4 mL/kg in the morning. Dosing solutions were made once at the start of the study. Heating at 56° C. and sonication were used to solubilize compound in the dosing solution. All procedures were done in concordance with ACUC guidelines. Terminal blood samples were obtained by cardiac puncture after CO₂ euthanasia and collected in EDTA tubes. Plasma was obtained after centrifugation. Brain tissue was dissected, weighed and frozen on dry ice until analysis. CSF samples were centrifuged to remove cells or debris prior to dilution at 1:2 in 4% BSA and frozen for subsequent analysis. Histopathological samples were placed in neutral buffered formalin prior to processing. Samples collected for occupancy were coated in embedding matrix, and frozen at −25° C. to −30° C. in a 2-methylbutane bath followed by dry ice. In life plasma samples were obtained using retro-orbital bleeding.

### Brain Abeta40 Assay

Rat brain (half a hemisphere) was homogenized using a polytron at 4 mL/g in PBS, pH 7.8, 2% CHAPS, complete protease inhibitors (Roche). Large debris was removed by centrifugation for 30 minutes at 20,800×g and the resulting supernatant was diluted 1:2 in PBS, 2.5% BSA. White Microlite II ELISA plates (Thermo Electron) were incubated with 50 μg/mL TSD9S3.2 antibody in PBS for 1 hour at 37° C. Plates were blocked with 200 μL 5% bovine serum albumin (BSA; weight/volume prepared in PBS) for 2 hours at room temperature on a plate shaker and then washed 5 times with 500 μL/well of PBS, 0.05% Tween-20. Clarified brain homogenates were loaded in 6 replicates of 50 μL per well and incubated for 1 hour at room temperature. Plates were washed as before and then incubated with horse radish peroxidase (HRP)-conjugated 252 antibody (Biosource) diluted 1:2000 in PBS, 0.05% Tween, 0.1% BSA for 1 hour. Three replicates contained the 252-HRP antibody only and three replicates contained the 252-HRP antibody with 1 μg/mL rat Aμ1-14 (Anaspec) which competed specifically bound antibody; this background signal was substrated from the total signal to yield the specific signal. The bound 252-HRP antibody was detected using Pierce Supersignal Pico Chemiluminescent substrate for 10 minutes and quantified a Packard TopCount. Samples were normalized to a brain homogenate reference placed on each plate. Based on Aβ40 standard curves, the LLQ was 10 pg/mL and the LLD was 20 pg/g tissue.

### Plasma Abeta40 Assay

Plates were coated with TSD antibody and washed as for the brain Aβ40 assay. Plasma samples were diluted 1:3 in PBS buffer, pH 7.8, 0.25% nonidet P40, 2.5% BSA. Samples were loaded in 6 replicates of 50 μL per well and incubated for 1-2 hours at room temperature. Samples were detected using 4G8-biotin (Signet) diluted 1:8000 in PBS, 0.05% Tween, 0.1% BSA for 1 hour. Three replicates had the 4G8-biotin antibody only and three replicates had the 4G8-biotin antibody with 1 μg/mL Aβ17-24 which competed the specific signal and thereby established a background value for each sample. Following washing as above, plates were incubated with streptavidin-HRP (Zymed) diluted 1:50,000 in PBS, 0.05% Tween, 0,1% BSA for 10 minutes. Detection and quantification were as for brain Aβ40 assays. Samples were normalized to a plasma reference placed on each plate. Based on Aβ40 standard curves, the LLQ was 7.5 pg/mL and the LLD was 23 pg/mL plasma.

### Acute Rat Studies

A single dose of the Compound of Example 7 at 10 mg/kg significantly reduced both plasma Aβ40 and brain Aβ40 to levels of 47% and 41% of vehicle control, respectively.

A γ-secretase inhibitor is considered active in one of the above in vivo assays if it reduces Aβ by at least 50% at a dosage of 100 mg/kg.

The above results confirm that compounds of the present invention are potent and selective γ-secretase inhibitors. These results support the use of compounds of the present invention as a therapeutic treatment for Alzheimer's disease, head trauma, traumatic brain injury, dementia pugilistica, and/or other disorders associated with β-amyloid peptide.

Therefore, the compounds of Formula I or pharmaceutical compositions thereof are useful in the treatment, alleviation or elimination of disorders or other disorders associated with the inhibition of β-amyloid peptide.

In another embodiment, this invention includes pharmaceutical compositions comprising at least one compound of Formula I in combination with a pharmaceutical adjuvant, carrier or diluent.

In still another embodiment, this invention relates to a method of treatment or prevention of disorders responsive to the inhibition of β-amyloid peptide in a mammal in need thereof, which comprises administering to said mammal a therapeutically effective amount of a compound of Formula I or a nontoxic pharmaceutically acceptable salt, solvate or hydrate thereof.

In yet another embodiment, this invention relates to a method for treating Alzheimer's Disease and Down's Syndrome in a mammal in need thereof, which comprises administering to said mammal a therapeutically effective amount of a compound of Formula I or a non-toxic pharmaceutically acceptable salt, solvate or hydrate thereof.

For therapeutic use, the pharmacologically active compounds of Formula I will normally be administered as a pharmaceutical composition comprising as the (or an) essential active ingredient at least one such compound in association with a solid or liquid pharmaceutically acceptable carrier and, optionally, with pharmaceutically acceptable adjuvants and excipients employing standard and conventional techniques.

The pharmaceutical compositions include suitable dosage forms for oral, parenteral (including subcutaneous, intramuscular, intradermal and intravenous) bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. The pharmaceutical compositions are prepared by conventional techniques appropriate to the desired preparation containing appropriate amounts of the active ingredient, that is, the compound of Formula I according to the invention. See, for example, Remington's Pharmaceutical Sciences, Mack Publishing Company, Easton, Pa., 17th edition, 1985.

The dosage of the compounds of Formula I to achieve a therapeutic effect will depend not only on such factors as the age, weight and sex of the patient and mode of administration, but also on the degree of β-AP inhibition desired and the potency of the particular compound being utilized for the particular disorder of disease concerned. It is also contemplated that the treatment and dosage of the particular compound may be administered in unit dosage form and that the unit dosage form would be adjusted accordingly by one skilled in the art to reflect the relative level of activity. The decision as to the particular dosage to be employed (and the number of times to be administered per day) is within the discretion of the physician, and may be varied by titration of the dosage to the particular circumstances of this invention to produce the desired therapeutic effect.

A suitable dose of a compound of Formula I or pharmaceutical composition thereof for a mammal, including man, suffering from, or likely to suffer from any condition related to β-AP production as described herein, generally the daily dose will be from about 0.05 mg/kg to about 10 mg/kg and preferably, about 0.1 to 2 mg/kg when administered parenterally. For oral administration, the dose may be in the range from about 1 to about 75 mg/kg and preferably from 0.1 to 10 mg/kg body weight. The active ingredient will preferably be administered in equal doses from one to four times a day. However, usually a small dosage is administered, and the dosage is gradually increased until the optimal dosage for the host under treatment is determined. In accordance with good clinical practice, it is preferred to administer the instant compounds at a concentration level that will produce an effective anti-amyloid effect without causing any harmful or untoward side effects. However, it will be understood that the amount of the compound actually administered will be determined by a physician, in the light of the relevant circumstances including the condition to be treated, the choice of compound of be administered, the chosen route of administration, the age, weight, and response of the individual patient, and the severity of the patient's symptoms.

The following examples are given by way of illustration and are not to be construed as limiting the invention in any way inasmuch as many variations of the invention are possible within the spirit of the invention.

### Synthesis

The compounds of the present application can be prepared in a number of ways well known to one skilled in the art of organic synthesis. The compounds of the present application can be synthesized using the methods described below, together with synthetic methods known in the art of synthetic organic chemistry, or variations thereon as appreciated by those skilled in the art. Preferred methods include, but are not limited to, those described below. All references cited herein are hereby incorporated in their entirety herein by reference.

The compounds may be prepared using the reactions and techniques described in this section. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents which are compatible with the reaction conditions will be readily apparent to one skilled in the art and alternate methods must then be used.

### General Reaction Schemes

The compound of the present invention can be prepared in a number of different ways well-known to one skilled in the art of organic synthesis. The compound of Formula I can be prepared by the methods described below in Reaction Scheme 1. Reasonable variations of the described procedures, together with synthetic methods which would be evident to one skilled in the art, are intended to be within the scope of the present invention.

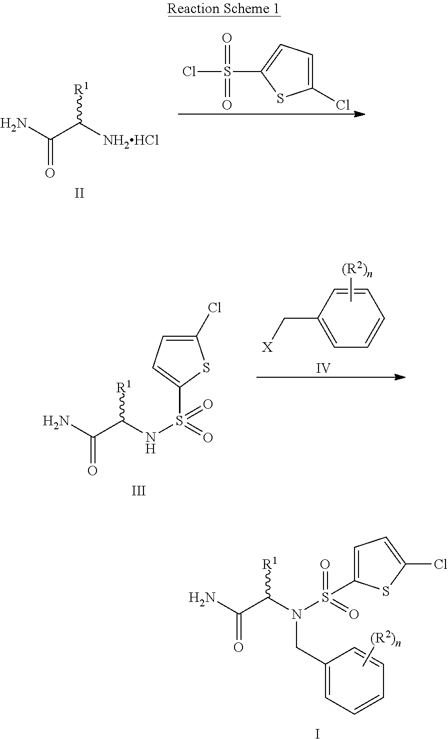

In one method of preparation illustrated in Reaction Scheme 1, the starting (α-amino)acetamide of Formula II in which R1 is 1,1,1-trifluoropropan-3-yl, which is used in substantially enantiomerically pure form, may be prepared by well-known literature procedures such as using the asymmetric Strecker synthesis method described in the Synthesis Section for the conversion of trifluorobutyraldehyde to the (α-amino)acetamide of Formula II in which R1 is 1,1,1-trifluorpropan-3-yl. The (α-amino)acetamide of Formula II in which R1 is cyclopropylmethyl may be obtained by conversion of the corresponding commercially available carboxylic acid to the primary amide by methods well known in the art. The (α-amino)acetamide of Formula II is treated with a suitable base and sulfonylated with 2-chlorothiophene sulphonyl chloride in a suitable aprotic solvent such as CH₂Cl₂ at about room temperature to afford the (α-sulfonamido)acetamide of Formula III. Suitable bases include triethylamine, diisopropylamine, pyridine and the like.

The conversion of the compound of Formula III to the sulfonamide of Formula I is carried out in the presence of a base by reacting the (α-sulfonamido)acetamide of Formula III with an alkylating agent of Formula IV in a suitable aprotic solvent with or without heating. The compound of Formula IV may readily be prepared by methods described in the Synthesis Section. Suitable bases for this alkylation include inorganic bases such as potassium carbonate and cesium carbonate. Preferred solvents include DMF and acetonitrile. The temperature range for the reaction is typically 20° C. to 100° C.

In the following examples, all temperatures are given in degrees Centigrade. Melting points were recorded on a Thomas Scientific Unimelt capillary melting point apparatus and are uncorrected. Proton magnetic resonance (¹H NMR) spectra were recorded on a Bruker Avance 300, a Bruker Avance 400, or a Bruker Avance 500 spectrometer. All spectra were determined in the solvents indicated and chemical shifts are reported in δ units downfield from the internal standard tetramethylsilane (TMS) and interproton coupling constants are reported in Hertz (Hz). Multiplicity patterns are designated as follows: s, singlet; d, doublet; t, triplet; q, quartet; in, multiplet; br, broad peak; dd, doublet of doublet; br d, broad doublet; dt, doublet of triplet; br s, broad singlet; dq, doublet of quartet. Infrared (IR) spectra using potassium bromide (KBr) or sodium chloride film were determined on a Jasco FT/IR-410 or a Perkin Elmer 2000 FT-IR spectrometer from 4000 cm⁻¹ to 400 cm⁻¹, calibrated to 1601 cm⁻¹ absorption of a polystyrene film and reported in reciprocal centimeters (cm⁻¹). Optical rotations [α]_{D} were determined on a Rudolph Scientific Autopol IV polarimeter in the solvents indicated; concentrations are given in mg/mL. Low resolution mass spectra (MS) and the apparent molecular (MH⁺) or (M−H)⁺ was determined on a Finnegan SSQ7000. High resolution mass spectra were determined on a Finnegan MAT900. Liquid chromatography (LC)/mass spectra were run on a Shimadzu LC coupled to a Water Micromass ZQ.

The following abbreviations are used: DMF (dimethylformamide); THF (tetrahydrofuran); DMSO (dimethylsulfoxide), Leu (leucine); TFA (trifluoroacetic acid); MTBE (methyltertbutylether); DAST [(diethylamino)sulfur trifluoride], HPLC (high pressure liquid chromatography); rt (room temperature); aq. (aqueous); AP (area percent).

### Synthesis

### A) Preparation of Intermediates

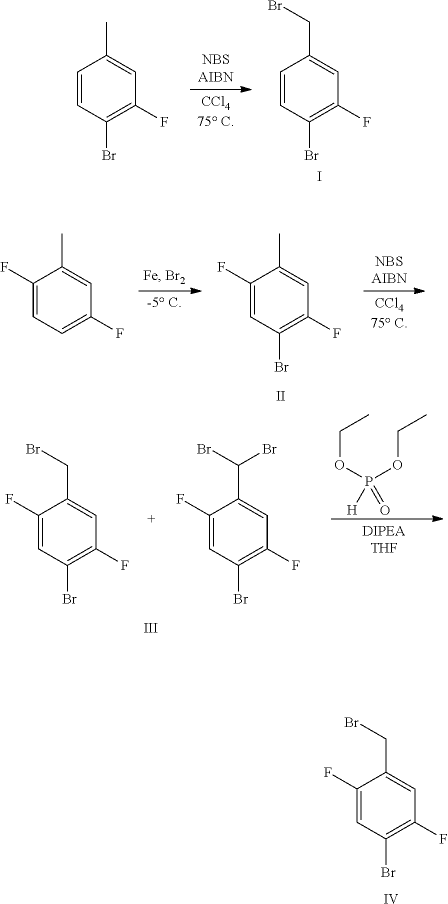

### (1) Preparation of Compound I

To a mixture of 4-bromo-3-fluorotoluene (5.0 g, 26.7 mmol) and N-bromosuccinimide (6.19 g, 34.8 mmol) in CCl₄ 34(50 mL) that was heated to 75° C. was added 2,2′-azobisisobutyronitrile (0.50 g, 3.0 mmol). The resultant mixture was stirred at 75° C. under nitrogen for 16 hours and then cooled to rt. The solvent was evaporated and the residue was dissolved in dichloromethane and filtered. Chromatography (silica gel, biotage, 40+M column, 2% to 30% EtOAc/Hexanes, 702 mL) provided the desired product as a pale yellow oil (4.3 g, 46% yield). ¹H NMR (DMSO-d⁶): δ 7.67 (d, J=2.5 Hz, 1H), 7.41 (d, J=8.5 Hz, 1H), 7.22 (d, J=8.5 Hz, 1H), 4.61 (s, 2H).

### (2) Preparation of Compound II

To a mixture of 2,5-difluorotoluene (2.5 g, 19.7 mmol) and iron powder (1.1 g, 19.7 mmol) was added bromine (2.8 g, 17.7 mmol) dropwise over a period of 30 min at −5° C. with mechanic stirring. The resultant mixture was stirred at −5° C. for 3 h and then EtOAc (20 mL) was added. Stirring was continued at rt for 5 min. and the precipitate was filtered off. The filtrate was concentrated and then purified by chromatography (silica gel, biotage, 40+M column, 0% to 10% EtOAc/Hexanes, 702 mL). A pale yellow oil was obtained.(3.2 g, 79.2% yield). LC/MS m/e: No (MH⁺), 1.99 min. 95% purity. ¹H NMR (DMSO-d⁶): δ 7.63 (dd, J=8.8, 6.0 Hz, 1H), 7.38 (dd, J=8.8, 6.8 Hz, 1H), 2.21 (s, 3H).

### (3) Preparation of Compound III

To a mixture of 2 (2.2 g, 10.7 mmol) and N-bromosuccinimide (3.2 g, 18.0 mmol) in CCl₄ (50 mL) that was heated to 75° C. was added 2,2′-azobisisobutyronitrile (0.60 g, 3.7 mmol). The resultant mixture was stirred at 75° C. under nitrogen for 16 hours and then cooled to rt. The solvent was evaporated and the residue was dissolved in dichloromethane, washed with water, dried over Na₂SO₄. Evaporation of solvent gave a pale yellow gum (4.0 g) that was directly used for next step.

### (4) Preparation of Compound IV

To a solution of III (4.0 g) and diethylphosphine (1.38 mL, 12.9 mmol) in THF (10 mL) was added diisopropylethylamine (2.0 mL, 11.5 mmol). The resultant mixture was stirred at rt for 2 h and then the precipitate was filtered off. The product was purified by chromatography (silica gel, biotage, 40+M column, 3% to 10% EtOAc/Hexanes, 702 mL). A pale yellow oil was obtained (1.2 g, 39.6% yield from 4-bromo-2,5-difluorotoluene II). LC/MS m/e: No (MH), 2.00 min. 79% purity. ¹H NMR (CDCl₃): 7.29 (dd, J=8.8, 5.6 Hz, 1H), 7.16 (dd, J=8.4, 6.8 Hz, 1H), 4.40 (s, 2H).

### EXAMPLE 1

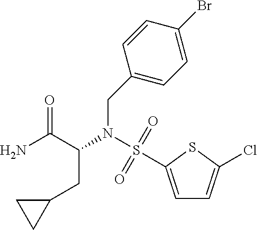

### Preparation of (R)-2-(N-(4-bromobenzyl)-5-chlorothiophene-2-sulfonamido)-3-cyclopropylpropanamide

To a solution of (R)-2-(5-chlorothiophene-2-sulfonamido)-3-cyclopropylpropanamide (100 mg, 0.32 mmol) and 4-bromobenzylbromide (120 mg, 0.48 mmol) in DMF (2 mL) was added Cs₂CO₃ (300 mg, 0.92 mmol). The resultant mixture was stirred at rt for 30 min and the precipitate was filtered off. The product was purified by chromatography (silica gel, biotage, 25+M column, 3% to 80% EtOAc/hexanes 702 mL) and was obtained as a white solid. (82 mg, 53.0% yield). LC/MS m/e: 477.0 (MH⁺), 2.38 min. 99% purity. ¹H NMR (DMSO-d⁶): δ 7.58 (s,1H), 7.56 (d, J=4.0 Hz, 1H), 7.52 (m, 1H), 7.49 (m, 1H), 7.36 (m, 1H), 7.33 (m, 1H), 7.27 (d, J=4.0 Hz, 1H), 7.17 (s, 1H), 4.77 (d, 3-17.0 Hz, 1H), 4.58 (d, J=17.0 Hz, 1H), 4.40 (t, J=7.0 Hz, 1H), 1.52 (m, 1H), 1.34 (m, 1H), 0.48 (m, 1H), 0.30 (m, 2H), 0.06 (m, 1H), −0.05 (m, 1H); HRMS (ESI): m/z calcd (C₁₇H₁₉N₂O₃BrClS₂) 476.9709, found 476.9697 [M+H]⁺.

### EXAMPLE 2

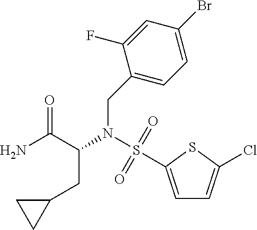

### Preparation of (R)-2-(N-(4-bromo-2-fluorobenzyl)-5-chlorothiophene-2-sulfonamido)-3-cyclopropylpropanamide

To a solution of (R)-2-(5-chlorothiophene-2-sulfonamido)-3-cyclopropylpropanamide (100 mg, 0.32 mmol) and 4-bromo-2-fluorobenzylbromide (120 mg, 0.45 mmol) in DMF (2 mL) was added Cs₂CO₃ (300 mg, 0.92 mmol). The resultant mixture was stirred at rt for 30 min and the precipitate was filtered off. The product was purified by chromatography (silica gel, biotage, 25+M column, 3% to 80% EtOAc/hexanes 702 mL) to obtained as a white solid. (95 mg, 56.0% yield). LC/MS m/e: 495.0 (MH⁺), 2.44 min. 99% purity. ¹H NMR (DMSO-d⁶): δ 7.62 (s,1H), 7.60 (d, J−4.0 Hz, 1H), 7.50 (m, 1H), 7.46 (m, 1H), 7.42 (m, 1H), 7.28 (d, J=4.0 Hz, 1H), 7.17 (s, 1H), 4.93 (d, J=17.0 Hz, 1H), 4.52 (d, J=17.0 Hz, 1H), 4.42 (t, J=7.0 Hz, 1H), 1.54 (m, 1H), 1.34 (m, 1H), 0.48 (m, 1H), 0.32 (m, 2H), 0.07 (m, 1H), −0.03 (m, 1H); ¹⁹F NMR (CD₃CN): δ −116.1; HRMS (ESI): m/z calcd (C₁₇H¹⁸N₂O₃BrFClS₂) 494.9615, found 474.9597 [M+H]⁺

### EXAMPLE 3

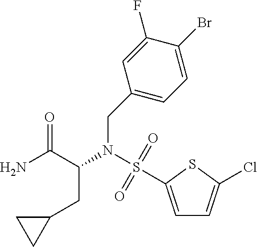

### Preparation of (R)-2-(N-(4-bromo-3-fluorobenzyl)-5-chlorothiophene-2-sulfonamido)-3-cyclopropylpropanamide

To a solution of (R)-2-(5-chlorothiophene-2-sulfonamido)-3-cyclopropylpropanamide (100 mg, 0.32 mmol) and 4-bromo-3-fluorobenzylbromide (120 mg, 0.45 mmol) in DMF (2 mL) was added Cs₂CO₃ (300 mg, 0.92 mmol). The resultant mixture was stirred at rt for 30 min and was filtered off precipitate. The product was purified by chromatography (silica gel, biotage, 25+M column, 3% to 80% EtOAc/hexanes 702 mL) to obtain a white solid. (94 mg, 59.3% yield). LC/MS m/e: 495.0 (MH³⁰), 2.38 min. 99% purity. ¹H NMR (DMSO-d⁶): δ 7.63 (m, 2H), 7.58 (d, J=4.3 Hz, 1H), 7.34 (dd, J=10.3, 1.7 Hz, 1H), 7.28 (d, J=4.0 Hz, 1H), 7.20 (m, 2H), 4.77 (d, J=17.0 Hz, 1H), 4.59 (d, J=17.0 Hz, 1H), 4.42 (t, J=7.0 Hz, 1H), 1.52 (m, 1H), 1.38 (m, 1H), 0.48 (m, 1H), 0.33 (m, 2H), 0.07 (m, 1H), −0.04 (m, 1H); ¹⁹ F NMR (CD₃CN): δ −109.33. HRMS (ESI): m/z calcd (C₁₇H₁₈N₂O₃BrFClS₂) 494.9615, found 474.9614 [M+H]⁺.

### EXAMPLE 4

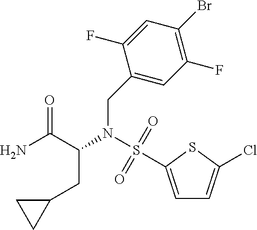

### Preparation of (R)-2-(N-(4-bromo-2,5-difluorobenzyl)-5-chlorothiophene-2-sulfonamido)-3-cyclopropylpropanamide

To a solution of (R)-2-(5-chlorothiophene-2-sulfonamido)-3-cyclopropylpropanamide (110 mg, 0.32 mmol) and 4-bromo-2,5-difiuorobenzylbromide (130 mg, 0.46 mmol) in DMF (2 mL) was added Cs₂CO₃ (300 mg, 0.92 mmol). The resultant mixture was stirred at rt for 45 min the precipitate was filtered off. The product was purified by chromatography (silica gel, biotage, 25+M column, 3% to 85% EtOAc/hexanes 702 mL) to afford a white solid. (78 mg, 42.6% yield). LC/MS m/e: 513.0 (MH⁺), 3.63 min. 96% purity. ¹H NMR (DMSO-d⁶): δ 7.69 (m, 2H), 7.61 (d, J=4.3 Hz, 1H), 7.38 (dd, J=10.3, 1.7 Hz, 1H), 7.31 (d, J=4.0 Hz, 1H), 7.23 (s, 1H), 4.87 (d, J=17.0 Hz, 1H), 4.53 (d, J=17.0 Hz, 1H), 4.43 (t, J=7.0 Hz, 1H), 1.52 (m, 1H), 1.42 (m, 1H), 0.50 (m, 1H), 0.33 (m, 2H), 0.07 (m, 1H), −0.03 (m, 1H); ¹⁹F NMR (CD₃CN): δ −114.46, −121.68. HRMS (ESI): m/z calcd (C₁₇H₁₇N₂O₃BrF₂ClS₂) 512.9521, found 512.9506 [M+H]⁺

### EXAMPLE 5

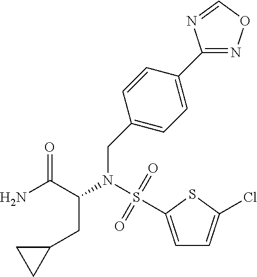

### Preparation of (R)-2-(N-(4-(1,2,4-oxadiazol-3-yl)benzyl)-5-chlorothiophene-2-sulfonamido)-3-cyclopropylpropanamide

To a solution of (R)-2-(5-chlorothiophene-2-sulfonamido)-3-cyclopropylpropanamide_(100 mg, 0.32 mmol) and 4-oxadiazolbenzylbromide (120 mg, 0.50 mmol) in DMF (2 mL) was added Cs₂CO₃ (300 mg, 0.92 mmol). The resultant mixture was stirred at rt for 30 min and the precipitate was filtered off. The product was purified by chromatography (silica gel, biotage, 25+M column, 3% to 80% EtOAc/hexanes 702 mL) to afford a white solid. (72 mg, 30.8% yield). LC/MS m/e: 477.0 (MH⁺), 2.38 min. 99% purity. ¹H NMR (DMSO-d⁶): δ 7.58 (s,1H), 7.56 (d, J=4.0 Hz, 1H), 7.52 (m, 1H), 7.49 (m, 1H), 7.36 (m, 1H), 7.33 (m, 1H), 7.27 (d, J=4.0 Hz, 1H), 7.17 (s, 1H), 4.77 (d, J=17.0 Hz, 1H), 4.58 (d, J=17.0 Hz, 1H), 4.40 (t, J=7.0 Hz, 1H), 1.52 (m, 1H), 1.34 (m, 1H), 0.48 (m, 1H), 0.30 (m, 2H), 0.06 (m, 1H), −0.05 (m, 1H); ¹⁹F NMR (CD₃CN): δ −66.4, −113.0-123.5. HRMS (EST): m/z calcd (C₁₇H₁₉N₂O₃BrCS₂) 476.9709, found 476.9697 [M+H]⁺.

### EXAMPLE 6

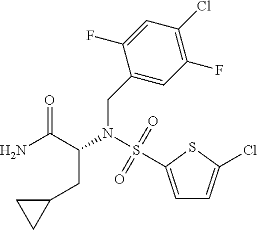

### Preparation of (R)-2-(5-chloro-N-(4-chloro-2,5-difluorobenzyl)thiaphene-2-sulfonamido)-3-cyclopropylpropanamide

Step A: To a solution of 4-chloro-2,5-difluorobenzoic acid (10 g, 52 mmol) in 100 ml diglyme was added NaBH4 (1.9 g, 50 mmol) slowly at RT to provide a white cloudy solution. BF3.2Et2O (7.0 ml, 55 mmol) was added dropwise over 1 to afford a clear solution. The mixture was stirred for an additional 3 h and quenched with 100 ml of ice water. The crude mixture was extracted with EtOAc and the organic layer was washed with brine and dried over Na2SO4. The solvent was evaporated to give 9.0 g of (4-chloro-2,5-difluorophenyl)methanol as an oil. MS (ES+, 161.07, 163.07, M-H2O). NMR (500 MHz, CDCl3) 7.2-7.3 (m, 1H), 7.0 (m, 1H), 4.5-4.7 (m, 2H).
Step B: To a solution of (4-chloro-2,5-difluorophenyl)methanol (9.0 g, 50.5 mmol) in 20 ml Et2O and 20 ml CH2Cl2 at RT was added PBr3 (1 ml, 10.6 mmol). The mixture was stirred for 10 minutes and then heated to 70 degree for 30 minutes. The mixture was cooled back to RT and poured into 30 ml water. The mixture was extracted with EtOAc, and the organic layer was washed with brine and dried over Na2SO4. The solvent was evaporated to afford 6.0 g of 1-(bromomethyl)-4-chloro-2,5-difluorobenzene as an oil (49%). NMR (500 MHz, CDCl3) 7.3-7.0 (m, 2H), 4.37 (s, 2H).

Step C: Preparation of Example 6.

To a solution of (R)-2-(5-chlorothiophene-2-sulfonamido)-3-cyclopropylpropanamide (110 mg, 0.36 mmol) and 4-chloro-2,5-difluorobenzylbromide (100 mg, 0.42 mmol) in DMF (2 mL) was added Cs₂CO₃ (300 mg, 0.92 mmol). The resultant mixture was stirred at rt for 30 min and the precipitate was filtered off. The product was purified by chromatography (silica gel, Biotage 25+M column, 3% to 80% EtOAc/hexanes 702 mL) to obtain 164 mg (0.35 mmol; 97% yield) as a white solid.
¹H NMR (300 MHz, CDCl3) δ ppm 7.4-7.3 (m, 2H), 7.1-7.0 (t, 1H), 6.93-6.91 (d, 1H), 6.17 (s, 1H), 5.25 (s, 1H), 4.65-4.50 (d, 1H), 4.4-4.3 (m, 2H), 1.83-1.80 (m, 1H), 1.27-1.22 (m, 1H), 0.44-0.30 (m, 3H), 0.1-0.2 (m, 2H). MS (LC/MS) [M+H]⁺=469.08 [M+Na]=491.06.

### Synthesis of Compound VI

### (R)-2-Amino-5,5,5-trifluoropentanamide hydrochloride

### Step A. 4,4,4-Trifluorobutanal

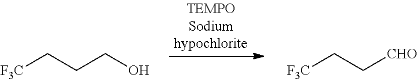

Dichloromethane (4.2 L) was charged into a 20 L four necked round bottom flask, equipped with mechanical stirring and cooling bath. The stirring was started and the reaction mixture cooled to 0 to −2° C. 4,4,4-Trifluorobutanol (750.0 g) was charged and the reaction mixture was cooled further to −5 to −8° C. TEMPO; (2,2,6,6-tetramethyl-1-piperidinyloxy, free radical) (9.15 g) was added while keeping the temperature between −5 to −8° C. An aqueous solution of potassium bromide (60 g in 1.17 L of water) was added to the above solution and the temperature was maintained at −5 to −8° C. An aqueous solution of NaOCl (8.8 L, 6-7% by wt., buffered to pH=8.5 using sodium bicarbonate) was added to the reaction mixture (caution: exothermic) while keeping the temperature of the reaction mixture at −5° C. Similarly, sodium periodate (NaIO₄) can substitute for NaOCl as the oxidizing agent. After complete addition, the dichloromethane layer was separated and the aqueous layer was washed with dichloromethane (1×750 mL). The dichloromethane layers were combined and dried using anhydrous sodium sulfate. The drying agent was filtered, and concentration of the solution of 4,4,4-trifluorobutanal was determined by NMR. The solution containing the title compound was used directly in the next step without additional processing. ¹H NMR (CDCl₃) (400 MHz) δ 2.30-2.50 (m, 2H, CH₂-CF₃), 2.70-2.80 (m, 2H, CH₂—CHO), 9.8 (s, 1H, CHO).

### Step B. 5,5,5-Trifluoro-2-(1-phenylethylamino)pentanenitrile (mixture of diastereomers)

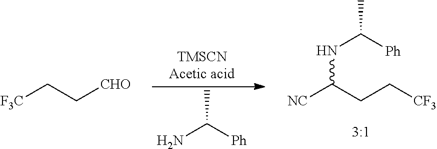

R-α-Methyl benzyl amine (528.5 g) was charged into a suitable vessel equipped with mechanical stirring, cooling bath and maintained under a blanket of nitrogen. 4,4,4-Trifluorobutyraldehyde solution (from Step A, 550 g) was charged, followed by methanol (3.3 L). The reaction mixture was then cooled to about 0 to −3° C. Acetic acid (glacial, 260 mL) was added drop-wise, maintaining the temperature around 0° C. followed by trimethylsilyl cyanide (581 mL) over a period of 15 minutes. Similarly, sodium cyanide (NaCN) or potassium cyanide could be used as the cyanide source. The reaction mixture was warmed to 25 to 27° C. and stirred overnight. Completion of the reaction was determined by TLC. Chilled Water (10.0 L) was charged into the reaction mixture and the reaction mixture was extracted with dichloromethane (1×10.0 L). The dichloromethane layer was washed with water (2×10.0 L) followed by brine (1×5.0 L). The dichloromethane layer was dried over anhydrous sodium sulfate and concentrated under reduced pressure to yield the title aminonitrile (mixture of diastereomers) as a viscous liquid, average yield 90%. ¹H NMR (CDCl₃) (400 MHz) δ 1.42 (d&m, 5H), 2.15 & 2.35 (two m, 1H each), 3.10-3.20 (m, 1H), 4.10-4.15 (m,1H), 7.10-7.35 (m, 6H).

### Step C. 5,5,5-Trifluoro-2-(1-phenylethylamino)pentanamide (mixture of diastereomers)

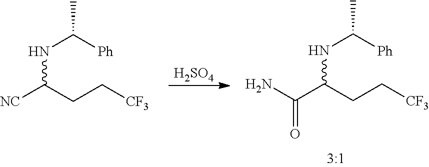

5,5,5-Trifluoro-2-(1-phenylethylamino)pentanenitrile (crude mixture of diastereomers from Step 13, 1.10 kg) was dissolved in dichloromethane (5.5 L) in a suitable vessel equipped with mechanical stirring, ice bath for cooling and maintained under a blanket of nitrogen. Stirring was started and the reaction mixture was cooled to 0 to −5° C. Concentrated sulfuric acid (1.75 L) was added dropwise over a period of 1 hour into the above mixture, maintaining the temperature below 0° C.; a clear solution was obtained after the addition was complete. The temperature of the reaction mixture was raised to 25 to 27° C. and stirred overnight (12-14H). Completion of the reaction was determined by HPLC. The reaction mixture was poured slowly over crushed ice (˜15.0 kg) and was neutralized with aqueous ammonia (˜25% by volume). The aqueous layer was separated and extracted with dichloromethane (2×3.0 L). The combined dichloromethane layer was washed with water (1×12.0 L) followed by brine (1×3.0 L). The product rich organic layer was dried over sodium sulfate and concentrated under reduced pressure to yield 0.85 kg (72.0%) of the crude title compound ¹H NMR (CDCl₃) (400 MHz) (Mixture of diasteromers) δ 1.36 (d&m, 4H, CH₃(J=8.0 Hz & 1H of CH₂), 1.90 (m, 1H of CH₂), 2.15 & 2.35 (two m, 1H each of CH₂—CF₃), 2.80-2.90 (m, 1H, CH-Ph), 3.60-3.70 (m,1H, —(CONH₂)CH(NH), 5.90 & 6.45 (1H each of CONH₂ with minor peaks for other diasteromer), 7.20-7.40 (m, 6H, Ar+NH).

### Step D. (R)-5,5,5-Trifluoro-2((R)-1-phenylethylamino)pentanamide hydrochloride

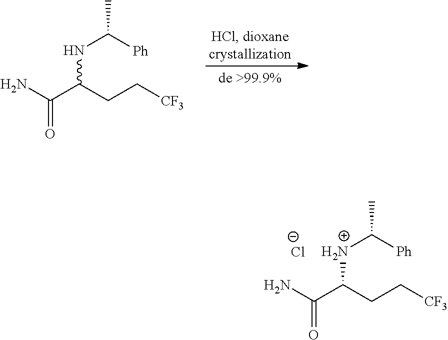

5,5,5-Trifluoro-2-(1-phenylethylamino)pentanamide (mixture of diastereomers) (850 g) was charged into a suitable vessel equipped with mechanical stirring and cooling bath. Methanol (2.55 L), ethyl acetate (1.7 L) and water (1.06 L) were charged and the reaction mixture was cooled to 0 to 5° C. A solution of HCl in dioxane (4.5 M, 1.72 L) was added dropwise over a period of 30 to 45 minutes. Similarly, mixtures of isopropanol and methyl tert-butyl ether could be used as solvent, and aqueous or concentrated HCl could be used as the HCl source. The temperature of the reaction mixture was then raised to 25 to 27° C. and stirred for 2 hours. Completion of the reaction was determined by TLC. The solid that precipitated was filtered and the cake was washed with a suitable solvent, such as ethyl acetate (1.8 L) followed by petroleum ether (2.5 L), or a mixture of isopropanol and methyl tert-butyl ether. The solid was allowed to dry at ambient temperature in an open tray, giving the title R-amino amide (480 g, 50% yield, diastereomeric excess=99.9%) ¹H NMR (CDCl₃) (400 MHz) δ 1.73 (d, 3H, CH₃, J=8.0 Hz), 2.08-2.09 (m, 2H of CH₂), 2.20-2.40 (m, 2H, CH₂—CF₃), 3.50-3.55 (m, 1H, CH-Ph), 4.40-4.41 (m,1H, —(CONH₂)CH(NH), 7.48-7.53 (br s, 5H, Ar).

### Step E. (R)-2-Amino-5,5,5-trifluoropentanamide hydrochloride (Compound VI)

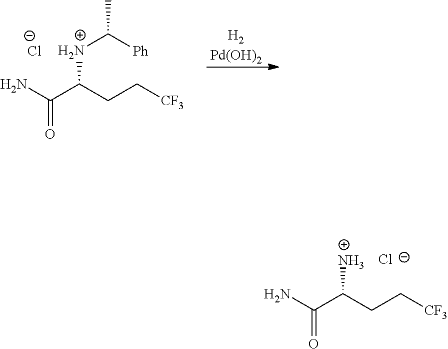

To a suitable pressure vessel, (R)-5,5,5-trifluoro-2-((R)-1-phenylethylamino)-pentanamide hydrochloride (1.50 kg) was charged along with methanol (15.0 L). This was followed by the addition of water (701.0 mL) followed by 20% palladium hydroxide on carbon (225 g). Similarly, palladium on carbon (Pd/C) could be used as the hydrogenation catalyst. The vessel was flushed with nitrogen three times, and then hydrogen gas was pressurized into the vessel (3-4 kg/cm2) at 60° C. The reaction was monitored for completion by HPLC. Upon completion, the reaction mixture was allowed to cool to 30-35° C. and filtered through a Celite pad, then washed with methanol. The filtrate was then concentrated under reduced pressure. After complete concentration, the remaining reaction mixture was treated with dichloromethane (2.5 L per wash), filtered and dried at 45° C. for 12 hours, giving the title compound (915 g, 91.0%; Purity=97%). ¹H NMR (DMSO-d₆) (400 MHz) δ 2.00 (m, 2H, CH₂), 2.30-2.40 (m, 2H of CH₂—CF₃), 3.85-3.88 (m,1H, —(CONH₂)CH(NH), 7.64 & 8.11 (br 5, 1H, each of CONH₂), 8.44 (br s, 3H, NH₃⁺). ¹³C NMR (DMSO-d₆) (100.0 MHz) δ 169.57, 131.20, 128.45, 125.71, 122.97, 50.91, 29.46, 29.18, 28.89, 28.61, 23.56, 23.53.

### Synthesis of Compound VII

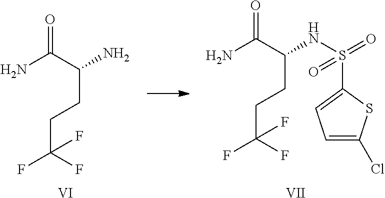

To a flask was added compound VI (2.0 grams, 9.7 mmol) in dichloromethane (100 ml). To this was added 5-chlorothiophene-2-sulfonyl chloride (3.0 grams, 13.9 mmol) and diisopropylethylamine (9 ml, 50 mmol). The reaction was allowed to stir at room temperature overnight. The reaction was then poured into Ethyl Acetate and water. The organic layer was extracted and washed with 0.1 N HCl, and saturated sodium chloride. The organic layer was concentrated in vacuo and then triturated in dichloromethane/hexanes to give 3.1 grams of compound VII. (91%). ¹H NMR (DMSO-d⁶): δ 8.4(d, 1H, J=8), 7.40-7.47(m, 2H), 7.15-7.30(m, 2H), 3.8-3.9(m, 1H), 2.0-2.3(m, 2H), 1.5-1.8(m, 2H).

### Synthesis of Compound VIII

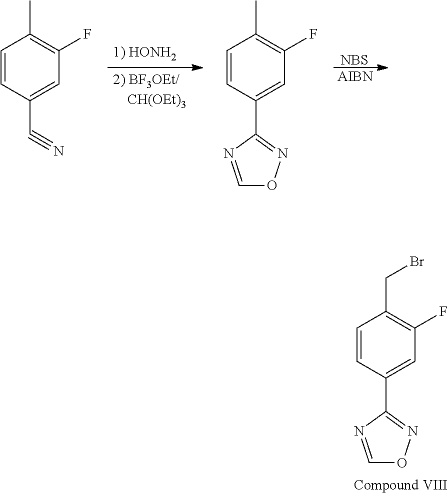

Step A: Into a 500 ml flask was added 3-fluoro-4-methylbenzonitrile (20 grams, 148 mmol) in ethanol (200 ml). To this was added hydroxylamine 50% in water (15 ml, 444 mmol). The reaction was heated to 80° C. for 1.5 hours and then allowed to cool to room temperature and concentrated to a residue. The residue was taken up in dichloromethane (200 ml). To this was added boron trifluoride etherate (1.2 mL) and triethyl orthoformate (60 mL). The reaction was then stirred at room temperature for 2 hours and then heated to reflux for 1 hour. After heating, the reaction was cooled to room temperature and purified on silca gel to obtain 24.2 grams (92%) of 3-(3-fluoro-4-methylphenyl)-1,2,4-oxidiazole. ¹HNMR (DMSO-d⁶): δ 7.76(d, 1H, J=12), 6.68(d, 1H, J=12), 7.47(t, 1H, J=8), 3.41 (s, 3H).
Step B: 3-(3-fluoro-4-methylphenyl)-1,2,4-oxidiazole (24 g, 134 mmol) was dissolved in carbon tetrachloride (200 ml). To this was added N-bromosuccimide (40 g) and AIBN (1.0 g). The reaction was heated to 80° C. for 18 hours and cooled to room temperature. The reaction was diluted with water and the organic layer was collected and concentrated. The residue was taken up in THF (60 ml) and diethyl phosphonate (12 mL) was added to the reaction, followed by diisopropylethylamine (18 ml). The reaction was allowed to stir at room temperature for 1 hour and then poured into water and ethyl acetate. The organic layer was collected and dried over magnesium sulfate and purified on a Biotage column, eluting with 5% ethyl acetate in hexanes. The product was collected and concentrated to yield 18.8 grams (53%, yellow solid) of Compound A. ¹H NMR (DMSO-d⁶): δ 9.77(s, 1H), 7.7-7.9(m, 3H), 4.76(s, 2H).

### Synthesis of Compound IX

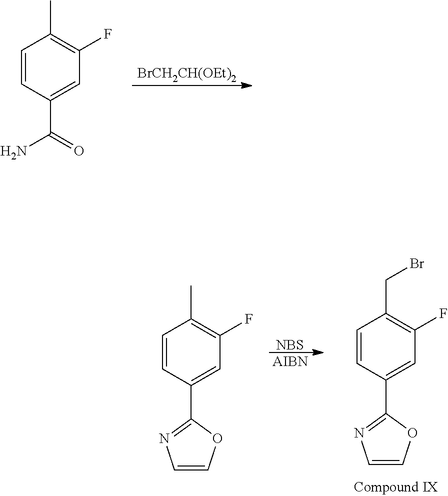

Step A Dissolved 3-fluoro-4-methylbenzamide (15.0 g, 97.9 mmol) in 75 mL of diglyme. To this was added 22 mL (146.9 mmol) of bromoacetaldehyde diethyl acetal and the mixture was heated at 130° C. for 8 hours. The mixture was cooled and diluted with ethyl acetate, washed with water (6×), 5% LiCl (3×), brine (2×). The organic layer was dried over magnesium sulfate, filtered and concentrated in vacuo. The crude material was purified in two batches on a 65M biotage column, eluting with 100% hexanes to 40% ethyl acetate/hexanes to afford 10.9 g of 2-(3-fluoro-4-methylphenyl)oxazole as a yellow solid (63% yield). 1H NMR (400 MHz, CHLOROFORM-d) δ ppm 7.68 (m, 3H), 7.25 (m, 2H), 2.31(d, 3H, J=1.8 Hz)
Step B: To a solution of 2-(3-fluoro-4-methylphenyl)oxazole (6.0 g; 33.9 mmol) in 160 mL of carbon tetrachloride was added N-bromosuccinamide (6.03 g; 33.9 mmol) and 2,2-azobisisobutyronitrile (556 mg; 3.39 mmol). The reaction was heated at 90° C. for one hour, cooled and filtered through a pad of celite. The filtrate was concentrated in vacuo, redissolved in ethyl acetate, and washed with water (3×) and brine (2×), then dried over magnesium sulfate, filtered and concentrated in vacuo. The residue was purified on a 65M biotage column, eluting with 5% ethyl acetate/hexanes to 55% ethyl acetate/hexanes to afford 5.77 g of Compound C (67%). 1H NMR (400 MHz, CHLOROFORM-d) δ ppm 7.82 (d, 1H), 7.80 (d, 1H), 7.74 (d, 1H), 7.72 (s, 1H), 7.47 (t, 1H, J=7.8 Hz), 4.52 (s, 2H).

### EXAMPLE 7

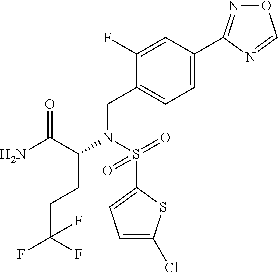

### Preparation of (R)-2-(5-chloro-N-(2-fluoro-4-(1,2,4-oxadiazol-3-yl)benzyl)thiophene-2-sulfonamido)-5,5,5-trifluoropentanamide

To a flask containing Compound VII (500 mg) in acetonitrile (25 ml) was added Compound VIII (500 mg) and CsCO3(1.0 g). The reaction was stirred at room temperature for 18 hours and then poured into ethyl acetate and water. The organic layer was then washed with saturated sodium bicarbonate, brine, and dried over magnesium sulfate. The organic layer was purified on a Biotage 25M column, eluting with 10-50% ethyl acetate in hexanes. The product was collected and concentrated to a residue. The residue was taken up in a minimal ethanol and 1 ml of ammonium hydroxide was added to the mixture. This was stirred at room temperature for 6 hours and re-purified on a Biotage 25M column, eluting with 10-50% ethyl acetate in hexanes. The compound was triturated with dichloromethane and hexanes. The precipitate was collected to yield 83 mg of a white powder (11% yield)

¹H NMR (DMSO-d⁶): δ 9.75(s, 1H), 7.9-8.0(m, 1H), 7.7-7.9(m, 2H), 7.6-7.7(m, 1H), 7.56(s, 1H), 7.3-7.4(m, 2H), 5.1(d, 1H, J=17), 4.7(d, 1H, J=17), 4.4-4.5(m, 1H), 2.0-2.2(m,2H), 1.9-2.0(m, 1H), 1.5-1.6(m, 1H). LC/MS (M+H) 526.99 at 2.493 mins (4.6×50 mm S10 column, 4 minute gradient).

### EXAMPLE 8

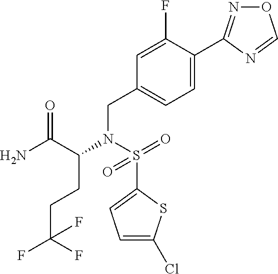

### Preparation of (R)-2-(5-chloro-N-(3-fluoro-4-(1,2,4-oxadiazol-3-yl)benzyl)thiophene-2-sulfonamido)-5,5,5-trifiuoropentanamide

Prepared 70 mg of Example 8 in 8% yield as a white powder in the same manner as Example 7, using compound IX. The final purification was performed via preparative HPLC on a 100×30 mm ID column @45 ml/min. ¹H NMR (DMSO-d⁶): δ 9.78(s, 1H), 8.0(t, 1H, J=8), 7.7(d, 1H, J=4), 7.6(s, 1H), 7.4-7.4(m, 2H), 7.4(s, 1H), 7.3(d, 1H, J=4), 4.86(d, 1H, J=20), 4.75(d, 1H, J=20), 4.47(t, 1H, J=8), 2.0-2.2(m, 2H), 1.8-2.0(m, 1H), 1.5-1.6(m, 1H). LC/MS (M+H) 526.99 at 2.227 minutes on a 4.6×50 mm S10, 4 minute gradient.

### EXAMPLE 9

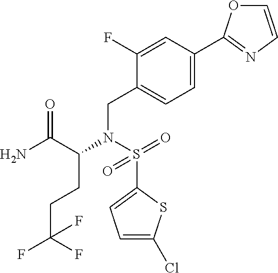

### Preparation of (R)-2-(5-chloro-N-(2-fluoro-4-(oxazol-2-yl)benzyl)thiophene-2-sulfonamido)-5,5,5-trifluoropentanamide

This compound was prepared in the same manner as Example 7 using 2-(4-(bromomethyl)-3-fluorophenyl)oxazole, yielding 602 mg (79%). The final purification was performed on a Biotage 40 column in the same solvents as Example 7. ¹H NMR (DMSO-d⁶): δ 8.26(s, 1H), 7.8-7.9(m, 1H), 7.6-7.8(m, 3H), 7.5(s, 1H), 7.4(s, 1H), 7.2-7.4(m, 2H), 5.0(d, 1H, J=20), 4.6(d, 1H, J=20), 4.5(t, 1, J=10), 2.0-2.3(m, 2H), 1.8-2.0(m, 1H), 1.5-1.7(m, 1H). LC/MS (M+H) 526.42 at 3.070 minutes on a 4.6×50 mm S10, 4 minute gradient.

### EXAMPLE 10

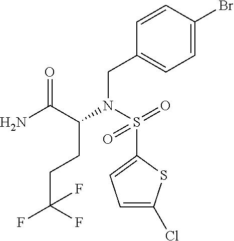

### Preparation of (R)-2-(N-(4-bromobenzyl)-5-chlorothiophene-2-sulfonamido)-5,5,5-trifluoropentanamnide

Dissolved compound VII (0.1 g, 0.28 mmol) and 1-bromo-4-(bromomethyl)benzene (0.085 g; 0.34 mmol) in 2 mL of DMF. Added CsCO3 (0.27 g; 0.84 mmol) and stirred for 2 hours; The mixture was poured into water and extracted with ethyl acetate (2×50 mL). The combined organic layer was washed with water and brine, dried over MgSO4 and stripped to dryness. The residue was taken up in methylene chloride and purified on a 40+ biotage column, eluting with 10% hexane/ethyl acetate to 50% hexane/ethyl acetate over 500 mL to afford 81 mg of the title compound as an oily solid (55% yield). HRMS cacld. For C₁₆H₁₄N₂O₃F₃S₂ClBr: 516.9270; Found: 516.9265. 1H NMR (400 MHz, CHLOROFORM-d) δ ppm 7.44 (2H, d, J=8.31 Hz), 7.33 (1H, d, J=4.03 Hz), 7.25 (1H, s), 7.23 (1H, s), 6.94 (1H, d, J=4.03 Hz), 6.08 (1H, br. s.), 5.23 (1H, br. s.) 4.43-4.55 (1H, m), 4.25-4.40 (2H, m), 1.98-2.23 (2H, m), 1.85 (1H, td, J=10.01, 4.41 Hz), 1.44-1.53 (1H, m). ¹⁹F NMR (400 MHz, CHLOROFORM-d) δ ppm −66.61. LC/MS phenomenex Luna 3×50 mm S10, 3 minute run, M+23=542 at 2.36 minutes.

### EXAMPLE 11

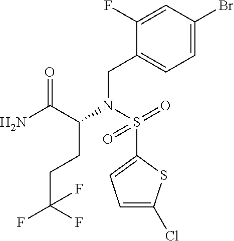

### Preparation of (R)-2-(N-(4-bromo-2-fluorobenzyl)-5-chlorothiophene-2-sulfonamido)-5,5,5-trifluoropentanamide

This compound was prepared in a similar manner to Example 11, using 4-bromo-1-(bromomethyl)-2-fluorobenzene, yielding 70% of the title compound. HRMS cacld. For C₁₆H₁₃N₂O₃F₄S₂ClBr: 534.9176; Found: 534.9169. 1H NMR (400 MHz, CHLOROFORM-d) δ ppm 7.31-7.42 (2H, m), 7.15-7.31 (3H, m), 6.95 (1H, d, J=4.03 Hz), 6.16 (1H, br. s.), 5.23 (1H, br. s.), 4.50-4.61 (1H, m), 4.31-4.43 (2H, m), 2.16-2.30 (1H, m), 1.99-2.16 (1H, m), 1.79-1.99(1H, m), 1.54-1.62 (1H, m). ¹⁹F NMR (400 MHz, CHLOROFORM-d) β ppm −66.60, −114.90. LC/MS phenomenex Luna 3×50 mm S10, 3 minute run, M+23=560 at 2.39 minutes.

### EXAMPLE 12

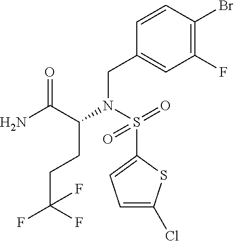

### Preparation of (R)-2-(N-(4-bromo-3-fluorobenzyl)-5-chlorothiophene-2-sulfonamido)-5,5,5-trifluoropentanamide

This compound was prepared in a similar manner to Example 10, using compound 1 described above, yielding 52% of the title compound. HRMS cacld. For C₁₆H₁₃N₂O₃F₄S₂ClBr: 534.9176; Found: 534.9161. 1H NMR (400 MHz, CHLOROFORM-d) δ ppm 7.45-7.53 (1H, m), 7.35 (1H, d, J=4.03 Hz), 7.17 (1H, dd, J=9.19, 1.89 Hz), 7.02 (1H, dd, J=8.18, 1.89 Hz), 6.95 (1H, d, J=4.03 Hz), 6.11 (1H, br. s.), 5.28 (1H, br. s.), 4.45-4.55 (1H, m), 4.27-4.41 (2H, m), 1.99-2.22 (2H, m), 1.86 (1H, ddd, J=14.98, 4.66, 4.53 Hz), 1.44-1.53 (1H, m). ¹⁹F NMR (400 MHz, CHLOROFORM-d) δ ppm −66.58. LC/MS phenomenex Luna 3×50 mm S10, 3 minute run, M+23=560 at 2.37 minutes.

What is claimed is:

1. A compound of formula I; or salt thereof 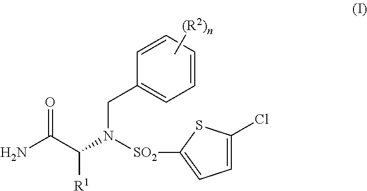
wherein:
R¹ is C₁₋₆ alkyl, —(CH₂)ᵣ—C₃₋₆ cycloalkyl, or C₁₋₆ haloalkyl;
R² is halo, oxadiazolyl, or oxazolyl;
n is 1 to 3; and
r is 1 to 3.

2. A compound of claim 1, wherein the compound is of formula (Ta) or salt thereof 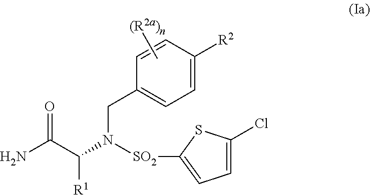
R² is halo, oxadiazolyl, or oxazolyl;
R^{2a} is halo;
n is 0-2.

3. A compound of claim 2, wherein
R¹ is C₁₋₄ alkyl, —(CH₂)ᵣ—C₃₋₆ cycloalkyl, or C₁₋₃ haloalkyl.

4. A compound of claim 3, wherein
R¹ is —(CH₂)—C₃₋₆ cycloalkyl, or C₁₋₃ haloalkyl.

5. A compound of claim 2, wherein
R² is halo,

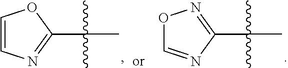

6. A compound of claim 5, wherein
R¹ is —(CH₂)-cyclopropyl, or —CH₂—CH₂—CF₃.

7. A compound of formula I; or salt thereof 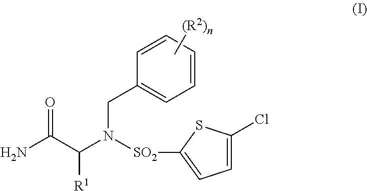
wherein:
R¹ is cyclopropylmethyl, or CF₃—CH₂—CH₂—;
(cycloalkyl —CH2-) or haloalkyl;
R² is halo, oxadiazolyl, or oxazolyl;
n is 1 to 3.

8. A compound of claim 7, wherein the compound is of formula (Ia) or salt thereof 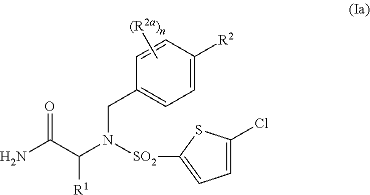
R² is halo, oxadiazolyl, or oxazolyl;
R^{2a} is halo;
n is 0-1.

9. A compound of claim 8, wherein
R² is halo,

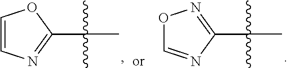

10. A compound of claim 7, wherein the compound is of formula (Ib) 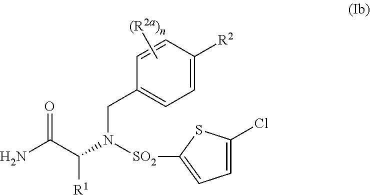
or salt thereof.

11. The compound of claim 1, wherein the compound is
(R)-2-(N-(4-bromobenzyl)-5-chlorothiophene-2-sulfonamido)-3-cyclopropylpropanamide;
(R)-2-(N-(4-bromo-2-fluorobenzyl)-5-chlorothiophene-2-sulfonamido)-3-cyclopropylpropanamide;
(R)-2-(N-(4-bromo-3-fluorobenzyl)-5-chlorothiophene-2-sulfonamido)-3-cyclopropylpropanamide;
(R)-2-(N-(4-bromo-2,5-difluorobenzyl)-5-chlorothiophene-2-sulfonamido)-3-cyclopropylpropanamide;
(R)-2-(N-(4-(1,2,4-oxadiazol-3-yl)benzyl)-5-chlorothiophene-2-sulfonamido)-3-cyclopropylpropanamide;
(R)-2-(5-chloro-N-(4-chloro-2,5-difluorobenzyl)thiophene-2-sulfonamido)-3-cyclopropylpropanamide;
(R)-2-(5-chloro-N-(2-fluoro-4-(1,2,4-oxadiazol-3-yl)benzyl)thiophene-2-sulfonamido)-5,5,5-trifluoropentanamide;
(R)-2-(5-chloro-N-(3-fluoro-4-(1,2,4-oxadiazol-3-yl)benzyl)thiophene-2-sulfonamido)-5,5,5-trifluoropentanamide;
(R)-2-(5-chloro-N-(2-fluoro-4-(oxazol-2-yl)benzyl)thiophene-2-sulfonamido)-5,5,5-trifluoropentanamide;
(R)-2-(N-(4-bromobenzyl)-5-chlorothiophene-2-sulfonamido)-5,5,5-trifluoropentanamide;
(R)-2-(N-(4-bromo-2-fluorobenzyl)-5-chlorothiophene-2-sulfonamido)-5,5,5-trifluoropentanamide; or
(R)-2-(N-(4-bromo-3-fluorobenzyl)-5-chlorothiophene-2-sulfonamido)-5,5,5-trifluoropentanamide;
or a salt thereof.

12. A pharmaceutical composition comprising a compound of claims 1-11 in association with a pharmaceutically acceptable adjuvant, carrier or diluent.

13. A method for the treatment or delaying the onset of Alzheimer's disease, cerebral amyloid angiopathy, mild cognitive impairment and/or Down syndrome which comprises administering to a mammal in need thereof a therapeutically effective amount of a compound of claims 1-11.

14. The method of claim 13 for the treatment of Alzheimer's disease.

15. A method for the treatment of head trauma, traumatic brain injury, and/or dementia pugilistica, which comprises administering to a mammal in need therof a therapeutically effective amount of a compound of claims 1-11.
